(19) United States
(12) Patent Application Publication
(10) Pub. No.: US 2013/0121967 A1
(43) Pub. Date: May 16, 2013

(54) BACTERIOPHAGES FOR USE AGAINST BACTERIAL INFECTIONS

(71) Applicant: Robert Leah, Birkerod (DK)

(72) Inventor: Robert Leah, Birkerod (DK)

(21) Appl. No.: 13/702,599

(22) PCT Filed: Jun. 8, 2011
(86) PCT No.: PCT/DK11/50201

§ 371 Date: Jan. 24, 2013

(30) Foreign Application Priority Data
Jun. 8, 2010 (DK) PA 2010 00492

(51) Int. Cl. A61K 35/76

(52) U.S. Cl. CPC A61K 35/76
USPC 424/93.2; 435/235.1

(57) ABSTRACT

The present invention relates to a composition comprising obligate lytic bacteriophages generated by a method comprising subjecting normally in vivo lysogenic, pseudolysogenic or temperate bacteriophages to genetic modifications in vitro, which alters the biological activity of one or more of the individual gene products for establishing, maintaining, controlling or regulating the lysogenic life cycle of the bacteriophages, thereby converting them to obligate lytic bacteriophages, wherein the genetic modification includes modification of a single gene in the operon containing a gene resulting in a gene product for establishing, maintaining, controlling or regulating the lysogenic life cycle of the bacteriophages.

### FIELD OF INVENTION

The present invention relates to the use of bacteriophages to combat bacterial infections; compositions containing mixtures of such bacteriophages directed and for use against a particular bacterial infection; methods of isolation, characterisation, and molecularly modification of such individual bacteriophages used in the mixtures rendering efficient clearance of particular bacterial pathogen infections.

### BACKGROUND OF THE INVENTION

Natural enemies of bacteria are the bacteriophages, which have been found virtually everywhere on the earth. Today, more than 5000 individual bacteriophages have been identified and catagorised into more than 250 genera, 60 families, and 3 orders, and the list is steadily increasing (Kutter et al., 2005). Bacteriophages only infect bacteria with no tropism against animals (humans) or plants.

The individual bacteriophage member, however, exhibit an extreme bacterial host specificity and infect and replicate in a very limited number of closely related bacteria.

Some bacteriophages exhibit a very hostile relationship on their target bacteria by executing a lytic bacteriophage life cycle, where the individual bacterium effectively is eliminated.

Lytic bacteriophages has for a long time been recognised as the ideal eliminator of bacteria in essence of their ability to kill the bacteria they infect.

However, most bacteriophages do not exhibit the feature of instant executing bacterial lysis, but rather enter a symbiotic relationship—the lysogenic cycle—where the genome of the bacteriophage integrates into the DNA of the bacterial host rendering the bacteriophage a ‘prophage’. This way, the bacteriophage's genomic material replicates along with the bacterial host without harming the life of the bacteria. The genetic material of the prophage is also reproduced when the bacteria reproduce.

When occassionally the conditions of the bacterial host deterioate, for example by stress due to depletion of nutrients, the prophage becomes active by initiating its own reproductive cycle, which ultimately render the bacterial host to lyse—ergo it behaves as a lytic bacteriophage.

Upon bacteriophage infection of its target bacteria, a choice is made—called the lysis-lysogen decision—where the bacteriophage either enters the lytic life cycle or the lysogenic life cycle.

The basis for this regulation is controlled by a delicate interactive balance between promoter and operator bacteriophage DNA sequences and a series of highly specific regulatory bacteriophage proteins rendering activities as repressors, modulators or activators directing gene expression of different sets of specific bacteriophage gene products.

The mechanisms of bacteriophage integration into the bacterial genome have been extensively studied and reviewed by Groth and Calos (2004). Of special interest has been the identification of bacteriophage integrases and their use as valuable genetic tools for genetic engineering of eukaryotic cells.

The integrase enzyme is a key mediator for lysogenic bacteriophages to accomplish integration of its genomic DNA into the bacterial host and thus entering a lysogenic life cycle.

An intermediate lifestyle of bacteriophages—pseudolysogeny describes a phage-bacterial host interaction in which no stable lysogen relationship is established (no integration of bacteriophage genetic material into the host genome) nor any production of new virions occur (as in a lytic response) Abedon (2008).

The genetic material of the bacteriophage simply resides within the bacterial host cell in a passive state without entering a replication cycle nor participates in segregation equally into all new bacterial progeny cells.

Establishing a pseudolysogenic state is an alternative strategy for both obligatory lytic and lysogenic bacteriophages to infect bacteria occasionally found in a poor physiological state, which may be unfavourable for commencing the bacteriophage's life cycle being either lytic or lysogenic.

As more favourable growth conditions occur, the production of new virions can be re-initiated for the normally obligate lytic bacteriophages or proceed with the process for establishing lysogeny for temperate bacteriophages.

The molecular mechanisms for controlling the pseudolysogeny is not known, but studies by Los et al. (2003) indicate that the rI gene product in bacteriophage T4 known to control the inhibition of lysis, also plays an important role in establishment and control of pseudolysogeny.

Acute bacterial pathogen infections in human are normally cleared by treatment by antibiotics with success. However, the successful outcome of such treatment is often hampered due to a steady escalating emergence of multi drug-resistant and pan-resistant bacterial pathogens.

In addition, formation of biofilm is a common cause of persistent infections—resistant to antibiotics or not—being associated to substantial morbidity and mortality not only in patients with systemic diseases—such as cystic fibrosis—, in patients with severe skin wounds, and in patients with urinary tract infections, but also in catheterised patients and in connection with medical implants (Costerton et al., 1999; Donlan, 2009).

The biofilm formation is associated with a profound phenotypic developmental change of the bacterial pathogen by transition from the free-floating planktonic growth phenotype to the biofilm stage, characterised with slow growth rates and embedding of the bacterial cells in a complex matrix of polysaccharides.

Combating pathogens embedded in biofilms by antibiotics and other antimicrobial medicaments are factors more difficult than elimination of the pathogen when in their planktonic growth stage.

In order to address possible antimicrobial activity against bacterial pathogen infections, animal infection models have been developed, covering aspects of either acute bacterial infections—being fast or slow growing—resistant to antibiotics—preferences for growth and development of biofilm persistence—in relation to relevant clinical indications. The thigh muscle infection model (Haller, 1985), the intraperitoneal foreign-body infection model (Christensen et al., 2007), the burn wound model (McVay et al., (2007), and the lung infection model (Song et al., (2005) have been used to study different aspects of Pseudomonas aeruginosa infection pathology and evaluations of possible therapeutics and their efficacy in vivo.

The concept of bacteriophage therapy has been well known for years and have been described in several publications, textbooks and reviews, for example by Sulakvelidze et al. (2001) and Kutter et al. (2005).

It has been well known in the art, that a key issue for approaching successful bacteriophage therapy to combat a bacterial pathogen infection, is that the bacteriophage applied exhibit effective lytic life cycle activity. Publications in the art describe isolation of specific bacteriophages with lytic activity against particular bacterial pathogens. These bacteriophages therefore are considered as possible candidates for therapeutic use against that particular bacterial pathogen. In fact, cocktails of different bacteriophages each with tropism against different bacterial pathogens have been state of the art concept for therapeutic use.

In nature, however, most bacteriophages are not obligate lytic bacteriophages but exhibit a lysogenic life cycle rendering them not suitable for therapeutic use. Furthermore, it is also recognised, that identification and isolation of lytic bacteriophages often is very-time consuming because lytic bacteriophages being effective against some bacterial pathogens are very rare and often hard to isolate.

Inventors of the EP1504088 (Rapson et al.) address this issue by applying a mutagenesis approach on a panel of lysogenic bacteriophages for identifying one or more “vir” mutants, a term the inventors define as mutations in an operator region of the bacteriophage DNA which prevent a repressor protein binding to the operator. The repressor protein normally binds to the operator so that the prophage is not transcribed and translated and does not enter the lytic cycle. However, the claimed “vir” mutants render the infecting bacteriophage DNA to be transcribed and translated and thus ultimately result in the lysis of the bacterial pathogen.

The complete genome sequences of at least 580 bacteriophages have been determined and filed in public genome data bases (eg. NCBI Entrez Genomes; http://www.ncbi.nlm.nih.gov/genomes). Isolation, purification and sequencing the genome of novel bacteriophages is a relative simple and effective task for a person skilled in the art. Furthermore, data mining of the novel bacteriophage's genome sequence enables identification of the possible gene products encoded. In addition, it also enables determination of their putative biological functions based on sequence homology analyses.

Molecular biology technologies, such as DNA subcloning, restriction endonuclease treatment, PCR amplification (Cheng et al., 1994), site directed mutagenesis, ligation technologies, and use of in vitro packaging extract technologies enable precise and specific DNA sequence modifications of virtually any nucleotide sequence within a bacteriophages genome.

The inactivation of a bacteriophage repressor, which transform a temperate bacteriophage into a lytic virion, has been disclosed previously (Lynch, K. H et al, 2010). According to this reference gene 41 of the bacteriophage KS9 was modified, producing a lytic mutant. The modification of the gene 41 included the integration by a single crossover of a resistance gene. The position of the crossover was not determined.

When performing a crossover it is uncertain at which position the DNA construct is integrated. In consequence, not only the repressor gene may be disrupted, but also further genes, such as genes regulated by the same promoter and/or operator may be affected. It is the object of the present invention to modify the genes of a bacteriophage to an extent that does not change the expression of further genes of the bacteriophage.

### DESCRIPTION OF THE INVENTION

The present invention relates to a composition comprising obligate lytic bacteriophages generated by a method comprising subjecting normally in vivo lysogenic, pseudolysogenic or temperate bacteriophages to genetic modifications in vitro, which alters the biological activity of one or more of the individual gene products for establishing, maintaining, controlling or regulating the lysogenic life cycle of the bacteriophages, thereby converting them to obligate lytic bacteriophages, wherein the genetic modification includes modification of a single gene in an operon containing a gene resulting in a gene product for establishing, maintaining, controlling or regulating the lysogenic life cycle of the bacteriophages.

The present invention uses a subset of the methods for modification of the DNA available to the skilled person, i.e. the subset of modifications suitable for effected the single gene of interest but not other genes upstream or downstream thereof. In a certain embodiment of the invention site-directed mutagenesis is preferred, especially when the genome of the bacteriophage is known wholly or partly. Site-directed mutagenesis, also called site-specific mutagenesis or oligonucleotide-directed mutagenesis, is a molecular biology technique in which a mutation is created at a defined site in a DNA molecule.

The basic procedure of site-directed mutagenesis requires the synthesis of a short DNA primer containing the desired base change. This synthetic primer has to hybridize with a single-stranded DNA containing the gene of interest. The single stranded fragment is then extended using a DNA polymerase, which copies the rest of the gene. The double stranded molecule thus obtained is then introduced into a host cell and cloned. Finally, mutants are selected.

The genetic modification may be any type of modification suitable of altering the original biological activity. In a certain embodiment a single nucleic acid is changed. This method is especially suitable when knowledge is available of invariant amino acids in the gene product. While a variety of methods are available for performing point mutation it is preferred to use PCR site-directed mutagenesis. Point mutations can be accomplished using polymerase chain reaction with oligonucleotide “primers” that contain the desired mutation. As the primers are the ends of newly-synthesized strands, by engineering a mis-match during the first cycle in binding the template DNA strand, a mutation can be introduced. Because PCR employs exponential growth, after a sufficient number of cycles the mutated fragment will be amplified sufficiently to separate from the original fragment by a technique such as gel electrophoresis, and reinstalled in the original context using standard recombinant molecular biology techniques.

When it is desired to change more than a single nucleic acid the genetic modification may be performed using the following steps: amplifying the nucleic acid fragment of the bacteriophage genome of interest,

subjecting the amplified DNA/RNA fragment to two separate PCR reactions, wherein a first PCR reaction uses a primer producing a PCR reaction product with an overhang DNA sequence and a second PCR reaction uses a primer producing a PCR reaction product with an overhang DNA sequence complementary to the overhang of the first PCR reaction product.

hybridizing the two PCR reaction products having overhang to each other.

extending the hybridized strands, thereby producing a DNA fragment, and

ligating the DNA fragment into the bacteriophage genome.

The overhang of the two PCR reaction products usually defines the modification of the gene. The gene products regulating the operation of an operon may be selected among the group comprising repressors, corepressors, activators, integrases, transposases, and transcriptional control proteins.

As used in the present description and claims an operon is defined as a functioning unit of genomic DNA containing a cluster of genes under the control of a single regulatory signal or promoter. The genes are transcribed together into an mRNA strand and either translated together in the cytoplasm, or undergo trans-splicing to create monocistronic mRNAs that are translated separately, i.e. several strands of mRNA that each encode a single gene product. The result of this is that the genes contained in the operon are either expressed together or not at all.

The inventor of the present invention have realized that it is of importance to maintain the production and the regulation of the gene products of the operon in which the mutated gene product for establishing, maintaining, controlling or regulating the lysogenic life cycle of the bacteriophages is encoded.

While the bacteriophages of the present composition may be targeted towards any bacterium, it is generally desired to design the obligate bacteriophages of the invention such that they are specific towards a certain bacterial pathogen, such as a human pathogen. The bacterial pathogen may cause a certain disease, such as infections caused by Pseudomonas aeruginosa. An other example is infections caused by bacteria of the genus Klebsiella, which causes various diseases such as pneumonia, urinary tract infections, septicemia, bacteremia, ankylosing spondylitis and soft tissue infections. Still another example include Acinetobacter, such as the species Acinetobacter baumannii, which is a key source of infections in debilitated patients in the hospital. While bacteria of the genus Escherichia generally are harmless to mammals, particular strains of some species are human pathogens and they are the most common source of urinary tract infections, significant sources of gastrointestinal diseases, ranging from simple diarrhea to dysentery-like conditions, as well as a wide-range of other pathogenic states.

The pathogen genus is generally selected among the group comprising enterococci, staphylococci, streptococci, enterobacter, bacteroides, escherichia, klebsiella, shigella, proteus, pseudomonas, salmonella, acinetobacter, citrobacter, helicobacter, propionibacterium, hemophili, mycobacteria, borrelia, neisseria, leptospirex and treponema.

In a certain aspect of the present invention the composition comprises a mixture of 2 or more different obligate lytic bacteriophages directed towards the same bacterial pathogen. It is presently believed that the chance of combating the bacterium is increased when combining two or more different obligate lytic bacteriophages. The mixture of obligate lytic bacteriophages each distinct from each other and belonging to either a similar or a different bacteriophage taxonomy group, all exert lytic activity towards the same bacterial pathogen target.

The amount of bacteriophages used in combating a certain disease may be of importance. Therefore, it is desired in an aspect of the invention that the titer of the one or more distinct lytic bacteriophages each is up to 10¹⁰ pfu/ml. In another aspect of the invention the titer of the one or more distinct lytic bacteriophages each is between 10¹⁰ and 10¹² pfu/ml, such as between 10¹⁰ and 10¹¹ pfu/ml or between 10¹¹ and 10¹² pfu/ml. In a most preferred aspect of the invention the titer of the one or more distinct lytic bacteriophages each is at least 10¹² pfu/ml.

The composition according to the invention may be formulated in various ways. Usually, the composition in addition to the obligate lytic bacteriophages contains an acceptable carrier. When intended as a human pharmaceutical, the carrier is pharmaceutically acceptable. Specific pharmaceutical formulations include a liquid, an aerosol, lyophilised powder, or adhered to acceptable nanoparticles.

The invention also accomplishes the use of the composition according to the present invention for combating bacterial pathogens. The composition may be administered in any fashion known to the skilled person, including administering through oral, nasal, ocular, intravenously, subcutaneus, transcutaneus, parental, intraperitoneal, rectal, vaginal, or topical applications.

The invention also relates to an obligate lytic bateriophage as such, which is generated by a method comprising subjecting a normally in vivo lysogenic, pseudolysogenic or temperate bacteriophage to genetic modification in vitro, which alters the biological activity of one or more of the individual gene products for establishing, maintaining, controlling or regulating the lysogenic life cycle of the bacteriophage, thereby converting the bacteriophage to an obligate lytic bacteriophage, wherein the genetic modification includes modification of a single gene in an operon containing a gene resulting in a gene product for establishing, maintaining, controlling or regulating the lysogenic life cycle of the bacteriophage.

Still another aspect of the present invention is the use of the obligate lytic bacteriophages for the manufacture of a pharmaceutical composition for combating a bacterial infection of a human.

Still another aspect of the invention relates to a method of producing an obligate lytic bacteriophage comprising the steps of

selecting an in vivo non-obligate lytic bacteriophage among the group consisting of lysogenic, pseudolysogenic, and temperate bacteriophages,

performing genetic modification in vitro of a gene resulting in altered biological activity of the gene products for establishing, maintaining, controlling or regulating the lysogenic life cycle of the bacteriophage, wherein

the genetic modification includes modification of a single gene in an operon containing a gene resulting in a gene product for establishing, maintaining, controlling or regulating the lysogenic life cycle of the bacteriophages.

In one aspect of the present invention a scalable pilot-scale or large-scale production and purification technology for manufacture of the individual obligate bacteriophages in the pharmaceutical composition is established for combating a bacterial infection in a human.

In a certain embodiment the production of the individual obligate bacteriophages to high yield is based on generation of liquid lysates in biofermentors, where the production host bacteria and bacteriophage are grown under suitable conditions and in a suitably defined medium.

In another aspect the production of the individual obligate bacteriophages to high yield is based on plate lysates, where the production host bacteria and bacteriophage are grown under suitable conditions and in a suitably defined semi-solid medium in large trays, thereby generating a lysate lawn of bacteriophages.

Following production of the bacteriophages, the crude liquid lysate or the collected slurry of the semi-solid lysate lawn may be subjected to an expanded bed adsorption chromatography using a suitable anion-binding ligand, such as DEAE (Diethylaminoethyl cellulose), for performing a combined capture, concentration, and IEX (Ion exchange chromatography) purification step. Following a combined buffer change, filtration and concentration step by tangential flow filtration, the bacteriophage preparation may further be purified by chromatographic removal of endotoxin using systems such as EndoTrap Blue (Lonza), followed by a sterile filtration by a 0.2 μm filtration for preparation of a pharmaceutical grade bacteriophage batch for the preparation of the composition according to the present invention.

### BRIEF DESCRIPTION OF THE FIGURES

FIG. 1 shows a general method for incorporating a modified sequence in a gene.

### SPECIFIC DISCLOSE OF THE INVENTION

The present invention includes the modification of a single gene in an operon. According to the present invention a gene is “operably linked” when it is placed into a functional relationship with another nucleic acid sequence. For example, DNA for a structural protein is operably linked to DNA for an activator if the structural protein is expressed together with the activator; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The term “primer” as used herein refers to an oligonucleotide, whether occurring naturally or produced synthetically, which is substantially complementary or homologous to all or part of a nucleic acid sequence to be amplified. The primer must be sufficiently long to hybridize with a template nucleic acid comprising the sequence to be amplified, and to prime the synthesis of an extension product in the presence of an agent for polymerization. Typically the primer will contain 15-30 or more nucleotides, although it may contain fewer nucleotides. It is not necessary, however, that the primer reflects the exact sequence of the nucleic acid sequence to be amplified or its complement. For example, non-complementary bases can be interspersed into the primer, or complementary bases deleted from the primer provided that the primer is capable of hybridizing with the nucleic acid sequence to be amplified or its complement, under the conditions chosen.

The term “obligate lytic bacteriophages” as used in the present description and claims is also referred to as virulent bacteriophages. Obligate lytic bacteriophages lack the ability of commencing a lysogenic phase. Instead, the obligate lytic bacteriophage multiply in the bacterial cell and causes the bacteria to burst. When the cell bursts the newly formed bacteriophages are liberated and ready for infecting new bacteria cells.

The bacterial pathogen to be combated according to the present invention may be selected from a variety of bacterial strains. Specific examples are: M. tuberculosis including multidrug-resistant variants of M. tuberculosis; Klebsiella pneumonia and Streptococci pneumonia and their MDR variants; Bordetella bronchoseptica as a cause of bronchopneumonia in animals and humans; Bordetella pertussis, a causative agent of acute and chronic respiratory infections and causing the major childhood disease whooping cough; Listeria monocytogenes, a causative agent of bacterial meningitis in infants; Salmonella typhimurium, a causative agent of typhoid fever in humans; Group B Streptococci, a causative agent for neonatal sepsis, pneumonia and meningitis; Salmonella enteritidis, a causative agent of within gastrointestinal and urogenital mucosa; Mycobacterium avium, the main pathogen of the M. avium complex that is a common opportunistic infection in AIDS patients; Legionella pneumophila, a causative agent for Legionnaires's disease; Leishmania donovani, a causative agent of leishmaniasis; Francisella tularensis, a causative agent of tularemia; Brucella abortus; Chlamydia spp. e.g. Chlamydia trachomatis; Rickettsia prowazekii; Shigella; Campylobacter; and Haemophilus influenzae type b, the major cause of bacterial meningitis in children.

A pharmaceutical composition according to the present invention may be suitable for administration to a patient in need thereof by way of oral, sublingual, transdermal or parenteral administration. In especially advantageous embodiments, said pharmaceutical composition may be suitable for administration by intranasal spray, by injection into peripheral blood vessels or by a intraperitoneal route.

For oral or parenteral administration, it is greatly preferred that the pharmaceutical composition is administered in the form of a unit-dose composition, such as a unit dose oral or parenteral composition. Such compositions are prepared by admixture and are suitably adapted for oral or parenteral administration, and as such may be in the form of tablets, capsules, oral preparations, powders, granules, lozenges, reconstitutable powders, injectable and liquid infusible solutions or suspensions or suppositories.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art.

Said composition may optionally include one or more additives, such as fillers, disintegrants, lubricants, wetting agents, and/or preservatives. Suitable fillers for use include cellulose, mannitol, lactose, trehalose and other similar agents. Suitable disintegrants include starch, polyvinylpyrrolidone and starch derivatives such as sodium starch glycolate. Suitable lubricants include, for example, magnesium stearate. Suitable pharmaceutically acceptable wetting agents include sodium lauryl sulphate. Suitable pharmaceutically acceptable preservatives include propyl p-hydroxybenzoate and sorbic acid.

These solid oral compositions may be prepared by conventional methods of blending, filling or tabletting. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral formulations also include conventional sustained release formulations, such as tablets or granules having an enteric coating.

For parenteral administration, fluid unit dose forms may be prepared comprising a sterile vehicle. The components of the composition, depending on the vehicle and the concentration, can be either suspended or dissolved. Parenteral solutions are normally prepared by dissolving the components of the composition in a vehicle and filter sterilising before filling into a suitable vial or ampoule and sealing. Advantageously, adjuvants such as a local anaesthetic, preservatives and buffering agents are also dissolved in the vehicle.

To enhance the stability, the composition may be frozen after filling into the vial and the water removed under vacuum.

Parenteral suspensions are prepared in substantially the same manner except that the compound may be suspended in the vehicle instead of being dissolved and sterilised by exposure to ethylene oxide before suspending in the sterile vehicle. Advantageously, a surfactant or wetting agent may be included in the composition to facilitate uniform distribution of the compound of the invention.

### EXAMPLES

### Purification of Primary Lytic Bacteriophages

Pre-treatment of potential bacteriophage containing samples:

1) Primary liquid samples are added 10×SM buffer (0.5M Tris-HCl (pH 7.5), 1 M NaCl, 0.1M MgSO₄) to final 1×SM buffer followed by filtration through a 0.45 um filter and stored in aliquots at 4-8° C.

2) Primary solid or semi-solid samples are chopped into fines, suspended in 10 volumes of 1×SM buffer, and incubated with stirring for up to 24 h at 4-8° C.

Following centrifugation, the cleared supernatant is filtered through 0.45 μm filter and stored in aliquots at 4-8° C.

In order to detect the presence of lytic bacteriophages in the samples, spot tests or plating are performed using different strains of targeting bacteria as the indicator host following incubation at different test growing conditions, such as different temperatures, CO₂ levels, or media compositions.

Cycles of Selective Amplification.

The sample may be subjected to one or more cycles of selective amplification by mixing a sample aliquot with the targeting bacteria, followed by addition of a growth media and incubation at selected test growing conditions. Following centrifugation, the cleared amplified supernatant is filtered through 0.45 μm filter and subjected to another cycle of selective amplification or tested for presence of lytic bacteriophages by the spot test or plating.

Isolation of Primary Lysogenic Bacteriophages.

Presence of Prophage.

Primary clinical isolates of the targeting bacterial pathogens are screened for harbouring lysogen bacteriophages by using mitomycin C as inducing agent.

The bacterial pathogen was cultured in growth media at selected test growing conditions until OD₆₀₀˜0.2. Half of the culture was transferred to a new tube and treated with Mitomycin C and both cultures were further incubated for up to 24 h. Cell lysis, indicated by clearance of culture after addition of Mitomycin C compared with the untreated control indicate presence of prophage which had switched to a lytic life cycle. This primary lysate is clarified by centrifugation followed by filtration through 0.45 μm filter and used for a second round of purification as follows:

A bacterial pathogen strain known not to contain prophages (see below) is used as indicator bacteria for infection with an aliquot of the primary lysate and plated at cell densities enabling isolation of individual bacteria after appropriate incubation time at selected test growth conditions. Individual bacteria are collected and screened for presence of lysogen bacteriophages as above. Occurence of cell lysis indicates presence of a bacteriophage in the primary lysate as a potential lysogen bacteriophage.

Absence of Prophage.

Bacterial pathogens not containing prophages will be used as indicator host for screening for primary lysogenic bacteriophages from primary sources.

In Vitro Modification of the Temperate Bacteriophage P2 Repressor Protein C.

Bacteriophage P2 is a representative of a group of temperate bacteriophage unrelated to bacteriophage A targeting Enterobacteriaeceae (Ljungquist et al., 1984). The bacteriophage P2-specific repressor gene is gene C, encoding a nonbasic gene product of 99 amino acids. The complete genome sequence of bacteriophage P2 (gene id: NC-001895) is determined to consist of 33593 by encoding a total of 43 putative gene products, all filed in public databases.

The C gene is located at position 5′-25890-->25591-3′ and the repressor protein C gene product of 99 amino acids (Id: PRO 149721) is shown to contain a DNA-binding motif positioned between aa positions 20 and 39 showing strong similarities to other DNA-binding regulatory proteins. The amino acid positions 24, 27, and 29 of the repressor protein C show very high degree of invariance between the different DNA-binding regulatory proteins.

This N-terminal amino acid region in the repressor protein C will therefore be subjected to in vitro targeted modification as depiched in this example in order to affect its capability to bind to the operator DNA sequence for exerting the repressor activity:

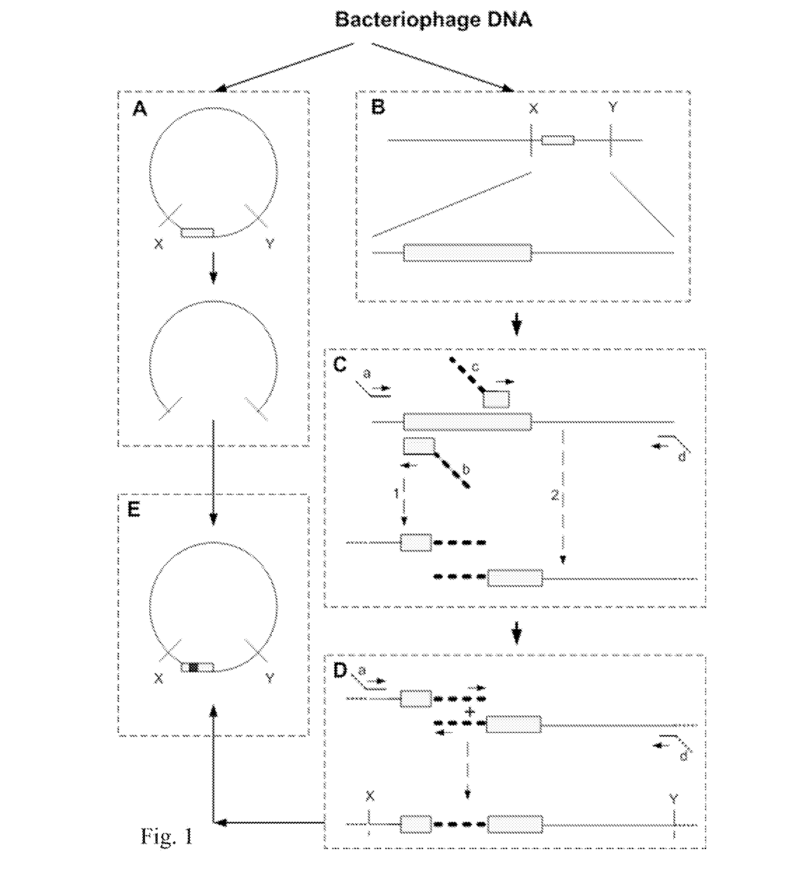

Isolated bacteriophage P2 DNA is subjected to restriction endonuclease treatment with two unique restriction enzymes Apol (pos. 25538) and EcoRV (pos. 30682) releasing a 5.1 kb DNA fragment containing the repressor protein C gene (see FIG. 1, B). The two unique restriction enzymes have been selected based on sequence analysis of the available bacteriophage P2 genome sequence.

The 5.1 kb DNA fragment is gel purified and subjected to two separate PCR reactions, one using the PCR primers a) and b) and one using PCR primers c) and d) (FIG. 1, C). The primers a) and d) have extensions for re-generating the Apol and EcoRV restriction site, respectively, while the primers b) and c) contains primer sequences 5′- to and -3′ to, respectively, the sequence for targeted in vitro modification (stipled lines), which encode the new sequence to be introduced by the in vitro modification.

For example, the sequence encoding the amino acids: ²⁴AlaAsp-Leu-Thr-Gly-Val²⁹, 5′-GCT GAT TTA ACA GGG GTT-3′, may be changed to 5′-GGG GAG ATT TTA GCT TTA-3′, resulting in the new amino acid sequence: ²⁴Gly-Glu-Ile-Leu-Ala-Leu²⁹.

Each of the resulting PCR fragments (1) and 2)) is gel purified and subjected to a combined denaturation, annealing and a first cycle PCR (polymerase) reaction (without outer primers), followed by addition of outer primers (primers a) and d), FIG. 1, D), subsequently finalising the PCR reaction amplification.

The resulting PCR product is subjected to restriction endonuclease treatment with Apol and EcoRV, and ligated back into the P2 DNA (FIG. 1, E) using a circularised P2 DNA (FIG. 1, A) restricted endonuclease treated with Apol and EcoRV followed by alkaline phosphatase treatment of fragment ends.

The resulting bacteriophage P2 DNA now contains an in vitro modified repressor C gene encoding a changed repressor C gene product with altered DNA-binding motif.

The DNA is transfected to the target bacterial pathogen or indicator bacteria (by CaCl₂ method or via in vitro packaging extract; Hohn and Hohn, 1974), and the readout is plaque formation on plates indicating a conversion of the preferable temperate P2 bacteriophage to an obligate lytic bacteriophage.

Modification In Vitro of Pseudomonas aeruginosa Bacteriophage MP22.

Bacteriophage MP22 is a representative of a temperate bacteriophage with a A-like morphology with tropism towards Pseudomonas aeroginosa (Heo et al., 2007). The complete genome sequence of the Bacteriophage MP22 (gene id: DQ-873690) is determined to consist of 36020 by encoding a total of 51 putative gene products.

The C repressor is located at position 5′-799-->170-3′ encoding a gene product of 209 amino acids (ID: YP1469130).

At position 5′-2059-->4131-3′, a gene product encoding a DNA transposition protein (transposase A) of 690 amino acids (ID: YP1469134) is identified.

Both the C repressor and the transposase A of the bacteriophage MP22 contain DNA-binding motifs at the N-terminal region.

The N-terminal amino acid region of either the C repressor or of the transposase A alone or in combination will be subjected to in vitro targeted modification by similar approaches as described for modification of the gene C of the bacteriophage P2 repressor.

In this case, the C repressor gene is contained in a unique 1.2 kb DNA fragment by treatment of the bacteriophage MP22 DNA with the two restriction enzymes SspI (pos. 140) and BsaI (pos. 1319) while the transposase A is contained in a unique 3.5 kb DNA fragment by treatment with the two restriction enzymes BsaI (pos. 1319) and SgrAI (pos. 4863).

Following basically the strategy as outlined for introducing the in vitro modification into the repressor C gene of the bacteriophage P2 (see FIG. 1), the modified DNA is transfected to the target bacterial pathogen or indicator bacteria (by CaCl₂ method or via in vitro packaging extract; Hohn and Hohn, 1974). The readout is plaque formation on plates indicating a conversion of the temperate bacteriophage MP22 to an obligate lytic bacteriophage.

Preparation of high titer bacteriophage samples and formulations for use in in vivo animal infection model testings will be according to protocols by Bujanover, U.S. Pat. No. 7,588,929). Test of the antimicrobial activity against bacterial pathogen infections by the bacteriophages will be performed by the different in vivo animal infection models for evaluation of antimicrobial potentials against both acute and persistent infections, to infections related to biofilm accumulation related to lung infections, wounds, use of indwelling medical devices and catheter-associated infection.

### REFERENCES CITED

Stephen T. Abedon (ed): Bacteriophage Ecology—Population Growth, Evolutioon, and Impact of Bacterial Viruses. Cambridge University Press (2008). ISBN: 978-0-521-85845-8.
Sergey Bujanover Production of bacteriophage compositions for use in phage therapy. U.S. Pat. No. 7,588,929.
Louise D. Christensen, Claus Moser, Peter Ø. Jensen, Thomas B. Rasmussen, Lars Christophersen, Staffan Kjelleberg, Naresh Kumar, Niels Høiby, Michael Givskov, and Thomas Bjarnsholt: Impact of Pseudomonas aeruginosa quorum sensing on biofilm persistence in an in vivo intraperitoneal foreign-body infection model. Microbiology Vol. 153, pp. 2312-2320 (2007).
J. W. Costerton, Philip S. Stewart, and E. P. Greenberg: Bacterial Biofilms: A Common Cause of Persistent Infections. Science Vol. 284, pp. 1318-1322 (1999).
Rodney M. Donlan: Preventing biofilms of clinical relevant organisms using bacteriophage. Trends in Microbiology Vol. 17, No. 2, pp. 66-72 (2009).
Cheng, S., Fockler, C., Barnes, W., Higuchi, R: Effective amplification of long targets from cloned inserts and human genomic DNA. Proc. Nat. Acad. Sci. USA Vol. 91, No. 12, pp. 5695-5699 (1994).
Amy C. Groth and Michele P. Calos: Phage Integrases: Biology and Applications. J. Mol. Biol. Vol. 335, pp. 667-678 (2004).
I. Haller: Comprehensive Evaluation of CiprofloxacinAminoglycoside Combinations against Enterobacteriaceae and Pseudomonas aeruginosa Strains. Antimicrobial Agents and Chemotherapy Vol. 28, No. 5, pp. 663-666 (1985).
Yun-Jeong Heo, In-Young Chung, Kelly B. Choi, Gee W. Lau and You-Hee Cho: Genome sequence comparison and superinfection between two related Pseudomonas aeruginosa phages, D3112 and MP22. Microbiology. Vol. 153, pp. 2885-2895 (2007).
B. Hohn and T. Hohn: Activity of Empty, Headlike Particles for Packaging of DNA of Bacteriophage X In Vitro. Proc. Nat. Acad. Sci. USA Vol. 71, No. 6, pp. 2372-2376 (1974).
Elisabeth Kutter and Alexander Sulakvelidze (Eds): BACTERIOPHAGES: Biology and Applications. CRC Press (2005). ISBN: 0-8493-1336-8.
Elisabeth Ljungquist, Kerstin Kockum, and L. Elizabeth Bertani: DNA sequences of the repressor gene and operator region of bacteriophage P2. Proc. Natl. Acad. Sci. USA. Vol. 81, pp. 3988-3992 (1984).
Marcin Los, Grzegorz Wegrzyn, and Peter Neubauer: A role for bacteriophage T4 rI gene function in the control of phage development during pseudolysogeny and in slowly growing host cells. Res. Microbiology Vol. 154, pp. 547-552 (2003).
Mark Edward Rapson, Faith Adelade Burden, Liudmilla Pencheva Glancey, David Allan Hodgson, and Nicholas Harold Mann: Bacteriophages useful for therapy and prophylaxis of bacterial infections. European Patent Specification EP1504088.
Zhijun Song, Hong Wu, Per Mygind, Dora Raventos, Carsten Sonksen, Hans-Henrik Kristensen, and Niels Høiby: Effect of Intratracheal Administration of Novispirin G10 on a Rat Model of Mucoid Pseudomonas aeruginosa Lung Infection. Antimicrobial Agents and Chemotherapy Vol. 49, No. 9, pp. 3868-3874 (2005).
Alexander Sulakvelidze, Zemphira Alavidze and J. Glenn Morris, Jr. Bacteriophage Therapy. Antimicrobial Agents and Chemotherapy Vol. 45, No. 3, pp. 649-659 (2001).
Catherine S. McVay, Marisela Velasquez, Joe A. Fralick: Phage Therapy of Pseudomonas aeuroginosa Infection in a Mouse Burn Wound Model. Antimicrobial Agents and Chemotherapy Vol. 51, No. 6, pp. 663-666 (2007).
Lynch h. Lynch, et al.: Inactivation of Burkholderia cepacia Complex Phage KS9 gp41 Identifies the phage repressor and generates lytic virions. Journal of Virology, Vol. 84, No. 3. p. 1276-1288 (2010).

1. A composition comprising obligate lytic bacteriophages generated by a method comprising subjecting normally in vivo lysogenic, pseudolysogenic or temperate bacteriophages to genetic modifications in vitro, which alters the biological activity of one or more of the individual gene products for establishing, maintaining, controlling or regulating the lysogenic life cycle of the bacteriophages, thereby converting them to obligate lytic bacteriophages, wherein the genetic modification includes modification of a single gene in an operon containing a gene resulting in a gene product for establishing, maintaining, controlling or regulating the lysogenic life cycle of the bacteriophages.

2. The composition according to claim 1, wherein the gene product having the biological activity altered is selected among the group comprising repressors, corepressors, activators, integrases, transposases, and transcriptional control proteins.

3. The composition according to claim 1, wherein the genetic modification is performed by the steps of:
amplifying the nucleic acid fragment of the bacteriophage genome of interest,
subjecting the amplified DNA/RNA fragment to two separate PCR reactions, wherein a first PCR reaction uses a primer producing a PCR reaction product with an overhang DNA sequence and a second PCR reaction uses a primer producing a PCR reaction product with an overhang DNA sequence complementary to the overhang of the first PCR reaction product,
hybridizing the two PCR reaction products having overhang to each other,
extending the hybridized strands, thereby producing a DNA fragment, and
ligating the DNA fragment into the bacteriophage genome.

4. The composition according to claim 3, wherein the overhang of the two PCR reaction products defines the modification of the gene.

5. The composition according to claim 1, the obligate bacteriophages are specific towards a bacterial pathogen.

6. The composition according to claim 5, wherein the bacterial pathogen in a human pathogen.

7. The composition according to claim 1, wherein the pathogen genus is selected among the group comprising enterococci, staphylococci, streptococci, enterobacter, bacteroides, escherichia, klebsiella, shigella, proteus, pseudomonas, salmonella, acinetobacter, citrobacter, helicobacter, propionibacterium, hemophili, mycobacteria, borrelia, neisseria, leptospirex and treponema.

8. The composition according to claim 1, wherein the bacterial pathogen is of a genus selected from Eescherichia, Acinetobacter, Pseudomonas and Klebsiella.

9. The composition according to claim 1, comprising a mixture of 2 or more different obligate lytic bacteriophages.

10. The composition according to claim 1, wherein the mixture of obligate lytic bacteriophages each distinct from each other and belonging to either a similar or a different bacteriophage taxonomy group, all exert lytic activity towards the same bacterial pathogen target.

11. The composition according to claim 1 wherein the titer of the one or more distinct lytic bacteriophages each is up to 10¹⁰ pfu/ml.

12. The composition according to claim 1, wherein the titer of the one or more distinct lytic bacteriophages each is between 10¹⁰ and 10¹² pfu/ml.

13. The composition according to claim 1, wherein the titer of the one or more distinct lytic bacteriophages each is at least 10¹² pfu/ml.

14. The composition according to claim 1, wherein the composition in addition to the obligate lytic bacteriophages contains an acceptable carrier.

15. The composition according to claim 1, for pharmaceutical use.

16. The composition according to claim 1, which is formulated into a liquid, an aerosol, lyophilised powder, or adhered to acceptable nanoparticles.

17. Use of a composition for combating bacterial pathogens, wherein the composition according to claim 1 is administered to a human infected with said bacterial pathogen, by virtue of administering through oral, nasal, ocular, intravenously, subcutaneus, transcutaneus, parental, intraperitioneal, rectal, vaginal, or topical applications.

18. An obligate lytic bacteriophage generated by a method comprising subjecting a normally in vivo lysogenic, pseudolysogenic or temperate bacteriophage to genetic modification in vitro, which alters the biological activity of one or more of the individual gene products for establishing, maintaining, controlling or regulating the lysogenic life cycle of the bacteriophage, thereby converting the bacteriophage to an obligate lytic bacteriophages, wherein the genetic modification includes modification of a single gene in an operon containing a gene resulting in a gene product for establishing, maintaining, controlling or regulating the lysogenic life cycle of the bacteriophages.

19. The obligate lytic bacteriophage according to claim 18, wherein the gene product having the biological activity altered, is selected among the group comprising repressor, activator, integrase, transposase, and transcriptional control protein.

20. The obligate lytic bacteriophage according to claim 18, wherein the genetic modification is performed by the steps of:
amplifying the nucleic acid fragment of the bacteriophage genome of interest,
subjecting the amplified DNA/RNA fragment to two separate PCR reactions, wherein a first PCR reaction uses a primer producing a PCR reaction product with an overhang DNA sequence and a second PCR reaction uses a primer producing a PCR reaction product with an overhang DNA sequence complementary to the overhang of the first PCR reaction product,
hybridizing the two PCR reaction products having overhang to each other,
extending the hybridized strands, thereby producing a DNA fragment, and
ligating the DNA fragment into the bacteriophage genome.

21. The obligate lytic bacteriophage according to claim 18, wherein the overhang of the two PCR reaction products defines the modification of the gene.

22. The obligate lytic bacteriophage to claim 18, which is specific towards a bacterial pathogen.

23. The obligate lytic bacteriophage according to claim 22, wherein the bacterial pathogen in a human pathogen.

24. The obligate lytic bacteriophage according to claim 18, wherein the pathogen genus is selected among the group comprising enterococci, staphylococci, streptococci, enterobacter, bacteroides, escherichia, klebsiella, shigella, proteus, pseudomonas, salmonella, acinetobacter, citrobacter, helicobacter, propionibacterium, hemophili, mycobacteria, borrelia, neisseria, leptospirex and treponema.

25. The obligate lytic bacteriophage according to claim 18, wherein the bacterial pathogen is of a genus selected from Escherichia, Acinetobacter, Pseudomonas and Klebsiella.

26. The obligate lytic bacteriophage according to claim 18, which is mixed with one or more different obligate lytic bacteriophages.

27. The obligate lytic bacteriophage according to claim 26, wherein the mixture of obligate lytic bacteriophages each distinct from each other and belonging either to a similar or a different bacteriophage taxonomy group, all exert lytic activity towards the same bacterial pathogen target.

28. Use of an obligate lytic bacteriophage according to claim 18, for the manufacture of a pharmaceutical composition for combating a bacterial infection of a mammal.

29. A method of producing an obligate lytic bacteriophage comprising the steps of:
selecting an in vivo non-obligate lytic bacteriophage among the group consisting of lysogenic, pseudolysogenic, and temperate bacteriophages,
performing genetic modification in vitro of a gene resulting in altered biological activity of the gene products for establishing, maintaining, controlling or regulating the lysogenic life cycle of the bacteriophage, wherein
the genetic modification includes modification of a single gene in an operon containing a gene resulting in a gene product for establishing, maintaining, controlling or regulating the lysogenic life cycle of the bacteriophages.

30. The method according to claim 29, wherein the gene product having the biological activity altered is selected among the group comprising repressors, activators, integrases, transposases, and transcriptional control proteins.

31. The method according to claim 29, wherein the genetic modification is performed by the steps of:
amplifying the nucleic acid fragment of the bacteriophage genome of interest,
subjecting the amplified DNA/RNA fragment to two separate PCR reactions, wherein a first PCR reaction uses a primer producing a PCR reaction product with an overhang DNA sequence and a second PCR reaction uses a primer producing a PCR reaction product with an overhang DNA sequence complementary to the overhang of the first PCR reaction product,
hybridizing the two PCR reaction products having overhang to each other,
extending the hybridized strands, thereby producing a DNA fragment, and
ligating the extended DNA fragment into the bacteriophage genome.

32. The method according to claim 29, wherein the overhang of the two PCR reaction products defines the modification of the gene.

33. The method according to claim 29, wherein the obligate bacteriophage is specific towards a bacterial pathogen.

34. The method according to claim 29, wherein the bacterial pathogen in a human pathogen.

35. The method according to claim 29, wherein the pathogen genus is selected among one or more of the group comprising enterococci, staphylococci, streptococci, enterobacter, bacteroides, escherichia, klebsiella, shigella, proteus, pseudomonas, salmonella, acinetobacter, citrobacter, helicobacter, propionibacterium, hemophili, mycobacteria, borrelia, neisseria, leptospirex and treponema.

36. The method according to claim 29, wherein the bacterial pathogen is of a genus selected from Escherichia, Acinetobacter, Pseudomonas and Klebsiella.
